(19) United States
(12) Patent Application Publication
(10) Pub. No.: US 2026/0060363 A1
(43) Pub. Date: Mar. 5, 2026

(54) APPARATUS AND SYSTEM FOR FOOTWEAR

(71) Applicant: Compedica Holdings Limited, Douglas, Isle Of Man (GB)

(72) Inventors: Daniel Blackman, Ickburgh (NF); James Mellon, Douglas Isle Of Man (GB); Denham Eke, Douglas Isle Of Man (GB); Jamie Gibson, Central (HK)

(21) Appl. No.: 18/819,682

(22) Filed: Aug. 29, 2024

(51) Int. Cl. A43B 3/44; A43B 7/00

(52) U.S. Cl. CPC A43B 3/44; A43B 7/00; A61B 5/1038; A61B 5/4833; A61B 5/68; A61B 5/6807; A61B 5/6812; A61B 17/1682; A61B 17/1775; G01C 22/006

(57) ABSTRACT

An apparatus for a footwear is described. An exemplary apparatus includes a sole adapted for insertion into a shoe wear worn on a foot of a person. The sole includes a sensor array disposed below a top surface of the sole. The sensor array is configured to generate sensor data for the foot of the person wearing the shoe wear. The sole includes a wireless communication unit and a processor disposed in a storage space in a bottom surface of the sole. The processor is in communication with the sensor array and the wireless communication unit. The processor is configured to receive the sensor data from the sensor array and communicate the sensor data to an external computing device through the wireless communication unit.

### TECHNICAL FIELD

The following disclosure is directed to footwear. In particular, the present disclosure is directed to apparatuses and systems of footwear.

### BACKGROUND

Patients with foot injuries may be given therapy regimes or instructions by a medical provider. However, recovery from these injuries can in-part depend on the patient's compliance to the provided therapy regimes and/or instructions. Accordingly, when the therapy regimes and/or instructions are not properly followed by the patient, the recovery time and discomfort may be prolonged.

### SUMMARY

In one aspect, an apparatus for footwear is disclosed. An apparatus can include a sole adapted for insertion into shoe wear. In some embodiments, the sole can include a sensor array disposed below the sole resting on a midsole of the shoe. The sensor array can be configured to generate sensor data of a shoe wearer (e.g., a patient). In further embodiments, the sole can include a wireless communication unit and a processor disposed in a storage space in a bottom surface of the sole. The processor can be in communication with the sensor array and the wireless communication unit. In some embodiments, the processor can be configured to receive the sensor data from the sensor array and communicate the sensor data to an external computing device through the wireless communication unit.

In another aspect, a system for footwear is presented. The system can include a sole adapted for insertion into shoe wear. The sole can include a sensor array disposed below a top surface of the sole. In some embodiments, the sensor array can be configured to generate sensor data of a shoe wearer (e.g., a patient). The sole can further include a wireless communication unit and a processor disposed in a storage space in the bottom surface of the sole. The processor can be in communication with the sensor array and the wireless communication unit. In some embodiments, the processor can be configured to receive the sensor data from the sensor array and communicate the sensor data to an external computing device through the wireless communication unit. The system can further include an offloading device having a twisted stem portion and a shin unit. In some implementations, the shin unit can be mechanically attached to the sole through the twisted stem portion, whose footplate section may be securely affixed to the midsole of the shoe which allows the wearer to immobilize tri-axis ankle movement of the foot at the subtalar joint.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from the foregoing and the following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 is a block diagram of a neural optogenetic implant, in accordance with some embodiments.

FIGS. 2A-B are illustrations of a sole of an apparatus for footwear, in accordance with some embodiments.

FIG. 3 is an illustration of a coupling between the sole of FIGS. 2A-B and a twisted stem portion, in accordance with some embodiments.

FIG. 4 is an illustration of a shin unit coupled to a twisted stem portion, in accordance with some embodiments.

FIG. 5 is a left side view of a shoe incorporating a system for footwear described herein, in accordance with some embodiments.

FIG. 6 is an illustration of a system for footwear in accordance with some embodiments.

FIG. 7 is a schematic representation of a fluidic circuit, in accordance with some embodiments.

FIG. 8 is a screenshot of a graphical user interface (GUI) showing a mapping of a patient's foot, in accordance with some embodiments.

FIGS. 9A-B are graphical user interfaces (GUIs) that can be implemented, in accordance with some embodiments.

FIG. 10 is a flowchart describing a method of remote patient monitoring, in accordance with some embodiments.

FIG. 11 is a block diagram of a machine learning model implemented with any apparatus or system described herein, in accordance with some embodiments.

### DETAILED DESCRIPTION

Patients with foot injuries and/or other conditions may be given therapy remedies, which may include rest and care instructions at home. Improper resting of a patient's foot and/or leg may cause injuries to worsen, and/or may prolong the patient's recovery times. Aspects of the present disclosure provide an apparatus that can record a patient's foot and/or leg use. In some embodiments, the apparatus described herein may provide compliancy tracking of a patient, which may ensure proper rest and recovery aligned with a therapy. Embodiments of the present disclosure may allow for remote patient monitoring with data being generated by the apparatus disclosed herein and communicated to a medical professional for interpretation via the cloud. In some embodiments, an on-board processor of the apparatus described herein may determine compliancy and/or other metrics of a patient's rest and recovery progress. In some embodiments, a system for footwear is provided herein, which may include a shin unit equipped with one or more speakers that may provide audible output to a patient regarding compliance, alerts, reminders, and the like.

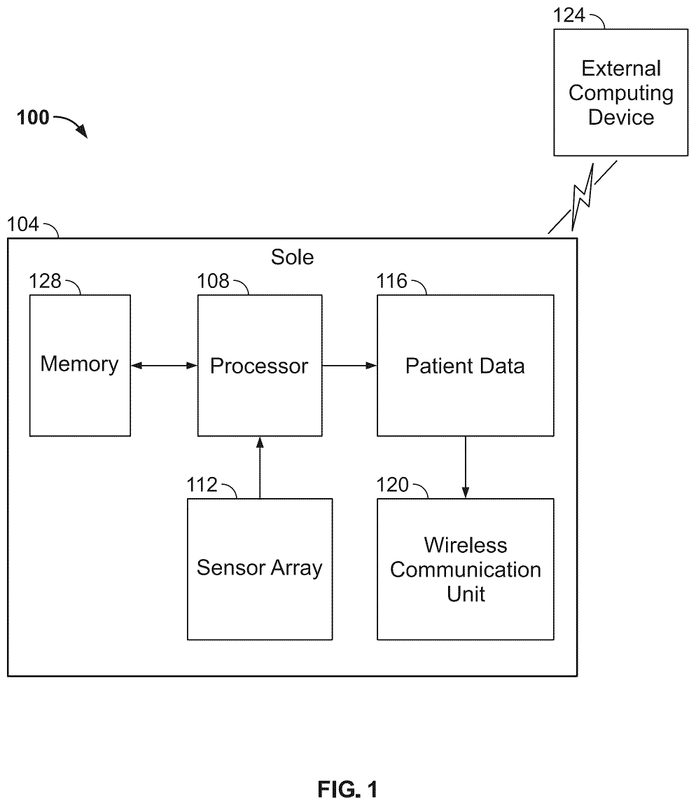

Referring now to FIG. 1, a block diagram of an apparatus 100 for footwear is presented. Apparatus 100 may include a sole 104. The term “sole” as used herein is an object placed underneath a person's foot in a shoe. Sole 104 may be shaped to fit in any size shoe and/or any type of shoe. Sole 104 can be designed to fit in a person's left shoe or right shoe. In some embodiments, sole 104 may be made in-part from a soft material, such as a fabric or gel-matrix.

Sole 104 may include a processor 108. Processor 108 may be any type of processor, such as a microcontroller, a System on a Chip (SoC), a microprocessor, and/or other types of processors. In some embodiments, processor 108 may be in communication with a memory, such as memory 128, having instructions thereon, which when executed by the processor 108, instruct the processor 108 to perform the various tasks described herein. Sole 104 may include a sensor array 112, which may include one or more sensors devices, thereafter referred to herein as sensors. A “sensor” as used herein is a device configured to detect a physical input and convert it into signals (e.g., digital signals) that can be measured and analyzed. One or more sensors of sensor array 112 may include, but are not limited to, pressure sensors, temperature sensors, humidity sensors, gyroscopes, and/or other types of sensors (e.g., light sensors). By way of example and not limitation, sensor array 112 may include 5 or more sensors. For instance and without limitation, sensor array 112 may include two sensors positioned on a rear end of sole 104 and three sensors positioned on a front end of sole 104. Sensor array 112 may be located just below a top surface of sole 104. In some embodiments, sensor array 112 may be located at a bottom surface of sole 104. Sensor array 112 may be configured to detect and generate sensor data. Sensor data may include, but is not limited to, pressure data, temperature data, acceleration data, force data, humidity data, positional data (e.g., an angle or rotation of a patient's foot, an angle of flexion of a patient's foot relative to a ground surface, an angle of relaxation of a patient's foot relative to a resting position, a change in angular positioning of a patient's foot, etc.), and/or other sensor information. Sensor array 112 may be configured to detect or calculate an number of steps taken by the person wearing the shoe over a period of time, according to some embodiments.

Sole 104 may include a wireless communication unit 120. A “wireless communication unit” as used herein is any device capable of transmitting electromagnetic signals. Wireless communication unit 120 may include, but is not limited to, a Bluetooth device, a Wi-Fi device, a cellular communications device, and/or other types of wireless communication units. Processor 108, which may be communicatively coupled to wireless communication unit 120, may be configured to communicate data to one or more external computing devices 124 via the wireless communication unit 120. External computing device 124 may be, for example, a smartphone, a tablet, a laptop computer, a desktop computer, a remote server, and/or other types of computing devices. In some embodiments, external computing device 124 may be a smartphone operated by the patient. In some embodiments, external computing device 124 may be a computing device operated by a medical professional, such as, but not limited to, nurses, doctors, physicians, and/or other medical professionals. Processor 108 may be configured to communicate raw sensor data generated by sensor array 112 to one or more external computing devices 124 via wireless communication unit 120. Raw sensor data may be unfiltered and/or uninterpreted data generated by sensor array 112. A medical professional may receive raw data generated by sensor array 112 at external computing device 124. A medical professional may provide feedback and additional instructions to the patient wearing sole 104 based on the sensor data received by the sensor array 112 of sole 104. For instance, a medical professional may determine that the patient is exerting excessive pressure on one or more parts of his/her foot and may instruct the patient to rest one or more parts of the foot for a period of time.

In some embodiments, processor 108 may be configured to receive sensor data generated by sensor array 112 and determine patient data 116. “Patient data” as used herein is any information relating to the patient. Patient data 116 may include, but is not limited to, various pressure readings/measurements of a patient's foot, position information of the patient's foot (e.g., one or more angles of a patient's foot in a resting position, one or more angles of a patient's foot while walking, average angle change in a patient's foot over a period of time, rotations of a patient's foot, etc.), number of steps taken by the patient over a period of time, geographic locations of the patient, periods of time the patient's foot is in contact with sole 104, and/or other information.

Processor 108 may be configured to determine a compliancy of a patient to a therapy. A “compliancy” as used herein can be a measure of alignment with a patient's actions and one or more parameters of a therapy regime. A “therapy regime”, also referred herein as a “recovery program,” refers to one or more parameters given to a patient to improve their diagnosis and/or symptoms thereof. Therapy regimes may include, for example, resting one or more parts of a patient's foot and/or leg for a period of time, exercising one or more parts of a patient's foot for a period of time, resistance exercises, walking a number of steps over a period of time, and/or other parameters. In some embodiments, processor 108 may receive information about one or more therapy regimes-such as reference data, instructions, and the like-from external computing devices 124. Processor 108 may be configured to compare sensor data generated by the one or more sensors of sensor array 112 with reference data from a therapy regime or other sources. Processor 108 may compare any type of data generated by sensor array 112, without limitation. For instance, processor 108 may compare an angular positioning of a patient's foot, pressure levels of various parts of a patient's foot, periods of rest of a patient's foot, temperature of a patient's foot, humidity of a patient's foot, number of steps taken over a period of time, and/or other data generated by sensor array 112 with stored reference data, reference threshold values, historical or other statistical data. A therapy regime may include, but is not limited to, reference pressure readings from one or more parts of the patient's foot, suggested number of steps taken by the patient over a period of time, suggested periods of rest time, periods of time that sole 104 can be in use, reference temperature levels of the patient's foot, and the like. In some embodiments, patient data 116 generated by sensor array 112 may be communicated holistically to a medical provider or a team of medical professionals, and the medical provider may draw inferences/determinations based on patient data 116 generated. As a non-limiting example, a medical provider may correlate a moderate pressure over a long period of time with a rise in temperature. Sensor array 112 may be calibrated to a specific sole 104, such that each sensor reading generated by sensor array 112 may be specific to the sole 104 and reference values used by processor 108 may be generated for the specific sole 104.

Processor 108 may be configured to quantify the patient's compliancy and to determine one or more levels of compliancy, percentage values of compliancy, scores out of 5, scores out of 10, or scores out of 100 by comparing, by way of example and not limitation, sensor data to one or more parameters, reference data, and reference threshold values of a therapy regime. Processor 108 may be configured to determine a compliance for each parameter of a plurality of parameters of a therapy regime. For instance, and without limitation, processor 108 may be configured to determine a compliancy score for a period of rest, pressure values for each part of a patient's foot, number of steps taken by the patient over a period of time, the amount of time sole 104 is in use, angular positioning of a patient's foot over a period of time, and/or other parameters. In some embodiments, processor 108 may be configured to generate a total compliancy score based on one or more parameters of a therapy regime. For instance, and without limitation, processor 108 may be configured to generate a total score out of 5, out of 100, a percentage value, and/or other metrics that may be used to identify levels of compliance. In some embodiments, processor 108 may determine one or more qualitative levels of compliance, such as, but not limited to, “non-compliant”, “semi-compliant”, “generally compliant”, “completely compliant”, and/or other levels. A “non-compliant” level may be determined based on a strong deviation from one or more parameters of a therapy regime and/or when the sensor data are above or below reference data and/or threshold values. A “completely compliant” level may be determined based on a strong adherence to one or more parameters of a therapy regime and/or when the sensor data are in agreement with reference data and/or threshold values. For instance, a patient may rest the exact amount given by a therapy regime, a patient may use sole 104 an exact amount given by a therapy regime, and/or a patient may apply various ranges of pressure to one or more parts of their foot identified by a therapy regime. Likewise, “semi-compliant” and “generally compliant” may be determined based on varying levels of adherence and deviation between “non-compliant” and “completely compliant” levels. In some embodiments, a total compliancy determined by processor 108 may be generated by giving weights to one or more parameters of a therapy regime. “Weights” as used herein refer to numerical values reflecting an importance of one or more variables. For instance, and without limitation, pressure of a patient's foot may be given a weight of 0.6, a temperature of a patient's foot may be given a weight of 0.1, and an angular positioning of a patient's foot may be given a weight of 0.3. Weights may be adjusted by processor 108 and/or may be provided by a medical professional via external computing device 124. In some embodiments, one or more parameters, reference data, and/or threshold values of a therapy regime may be adjusted over the course of time, such as by a medical professional, through external computing device 124. In other embodiments, processor 108 may be configured to automatically update and/or modify one or more parameters, reference data, and/or threshold values of a therapy regime based on sensor data generated by the one or more sensors of sensor array 112.

In some embodiments, processor 108 may be configured to generate a pressure multi-zone mapping of a patient's foot. A “pressure multi-zone mapping” as used herein is a visual pressure distribution analysis of two or more portions of a patient's foot. For instance, based on pressure data generated by sensor array 112, processor 108 may be configured to generate a pressure mapping of two or more zones of a patient's foot. In some embodiments, processor 108 may be configured to generate a pressure mapping of five separate zones of a patient's foot. Each zone of a pressure multi-zone mapping of a patient's foot may correspond to a particular bone and/or bone group of a patient's foot. For instance, a first zone may correspond to the distal phalanges, the second zone may correspond to the middle phalanges, the third zone may correspond to the proximal phalanges, the fourth zone may correspond to the metatarsal bones, and the fifth zone may correspond to the tarsus. In some embodiments, each zone may correspond to a particular muscle group, such as, but not limited to, the flexor hallucis brevis, the abductor hallucis, the quadratus plantae, the lumbricals, the abductor digiti minimi, the flexor digitorum brevis, and/or any other muscles in the foot and/or ankle. Processor 108 may be further configured to generate a visual representation of a pressure multi-zone mapping. By way of example and not limitation, processor 108 may be configured to generate a color coded pressure mapping overlaid on a visual representation of a patient's foot. A color coded pressure mapping may include red for high pressure areas, orange for semi-high pressure areas, yellow for medium pressure areas, and/or green for low pressure areas. In some embodiments, a visual pressure mapping of multiple zones of a patient's foot may include pressure values displayed next to each portion of the patient's foot. In some embodiments, processor 108 may be configured to determine if sole 104 is being worn properly via data generated by the sensor array 112, such as patient data 116. For instance, patient data 116 may include pressure data, which processor 108 may interpret to determine whether a patient is wearing sole 104 as instructed (i.e., properly). By way of example and not limitation, processor 108 may compare sensor data to one or more reference values, and based on the comparison, may determine whether a patient is wearing sole 104 as instructed. Reference values may, for example, be provided via external input to processor 108 through wireless communication unit 120. In some embodiments, processor 108 may generate reference values for a specific patient, such as during a start up or initialization process. For example, in an initialization process, a patient may place his/her foot on sole 104 for a period of time and processor 108 may generate one or more reference values for the patient, such as pressure values, pressure mapping of the patient's foot, temperature values, and/or other data. Processor 108 may be further configured to determine, based on sensor data generated by sensor array 112, whether sole 104 operates according to specifications. For instance, processor 108 may determine whether any of the elements, modules, and/or units in sole 104 malfunctions. For example, processor 108 may run one or more tests for the elements, modules, and/or units in sole 104 to determine their operational status.

Alternatively, processor 108 may be configured to collect and send the raw pressure data generated by sensor array 112 to an external computing device 124 where the raw pressure data can be converted to a pressure multi-zone mapping of the patient's foot with the characteristics noted above.

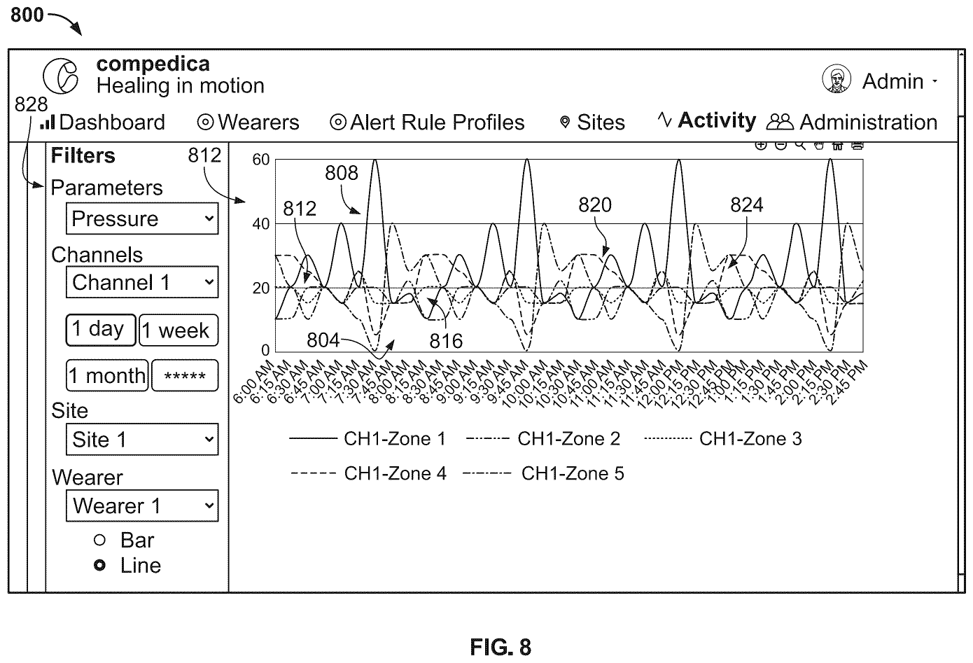

Still referring to FIG. 1, in some embodiments, processor 108 and/or external computing device 124 may be configured to run one or more machine learning models. A machine learning model may described in further detail below with reference to FIG. 8. A machine learning model may be trained with training data correlating sensor data to one or more levels of compliance, one or more levels of predicted compliancy, or other inputs. Training data for a machine learning model may be received via user input, external computing devices, and/or previous iterations of processing. In some embodiments, a machine learning model may be trained and it's parameters may be communicate to processor 108, which may off-load the computational heavy task of training and running the machine learning model elsewhere (e.g., to the external computing device 124). In some embodiments, a machine learning model may be run exclusively on external computing device 124. Processor 108 and/or external computing device 124 may utilize a machine learning model to interpret the vast amount of data generated by sensor array 112. For instance, and without limitation, a machine learning model running on processor 108 and/or external computing device 124 may use sensor data generated by sensor array 112 as input data, and may provide one or more compliance scores and/or compliance predictions as output data. For instance, a machine learning model may predict how a patient complies with a therapy regime. In some embodiments, a machine learning model may be trained with training data correlating sensor data to predicted levels of improvement and/or recovery of a diagnosis and/or symptom of a patient. Training data correlating sensor data to predicted levels of improvement and/or recovery may be received via user input, external computing devices, and/or previous iterations of processing. A machine learning model running on processor 108 and/or external computing device 124 may be configured to input sensor data generated by sensor array 112 and output estimated timelines for a full recovery for a patient, such as, but not limited to, days, weeks, months, and the like.

In some embodiments, processor 108 may utilize a classifier as described below with reference to FIG. 8. A classifier utilized by processor 108 may be trained with training data correlating sensor data and therapy regime parameters to levels of compliancy. Training data may be received via user input, external computing devices, and/or previous iterations of processing. A classifier run by processor 108 may be configured to input sensor data and, based on one or more parameters of a therapy regime, output one or more levels of compliancy. Levels of compliancy may include “non-compliant”, “semi-compliant”, “generally compliant”, “completely compliant”, and/or other levels, as noted above.

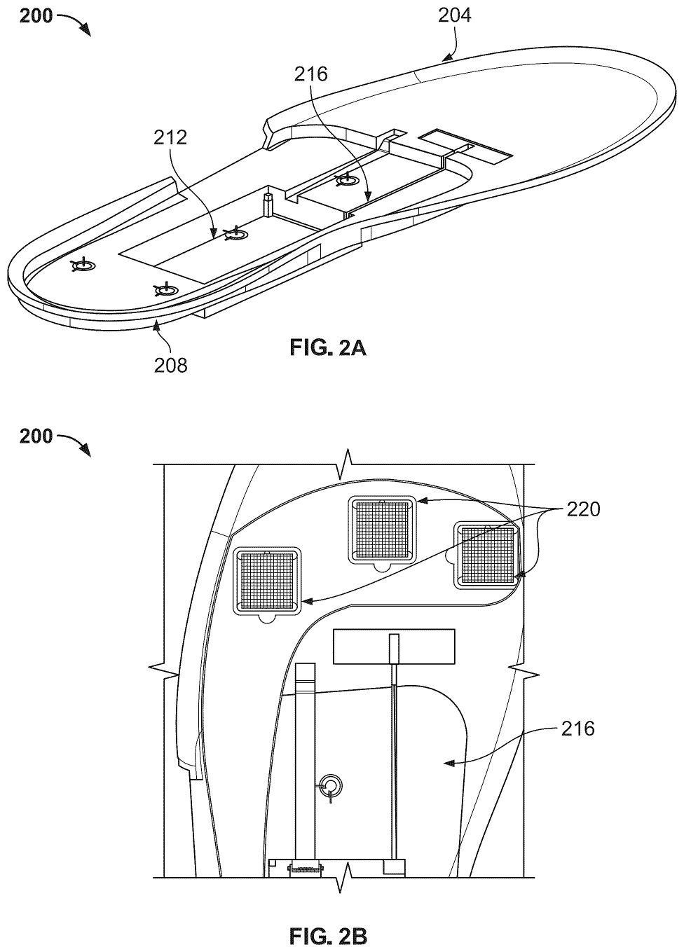

Referring now to FIG. 2A, a side perspective view of sole 200 is presented. According to some embodiments, sole 200 may correspond to or represent sole 104 discussed above in connection to FIG. 1. Sole 200 may include a top surface 204 and a bottom surface 208, positioned opposite top surface 204. Top surface 204 may be designed to come in contact with the foot of a patient. In some embodiments, bottom surface 208 may be designed to contact a bottom of a shoe to which the sole 200 is inserted. Sole 200 may include a storage compartment 212. Storage compartment 212 may be an indented portion of sole 200 (e.g., a shallow cavity). For instance, storage compartment 212 may be a rectangular, circular, hexagonal, or other appropriately shaped compartment that may connect the top surface 204 to an interior of bottom surface 208. Storage compartment 212 may store one or more components, such as, but not limited to, processors, memories, power sources, wireless communication units, and/or other components, such as those discussed above in reference to sole 104. In some embodiments, sole 200 may include one or more electrical connection pathways 216. Electrical connection pathways 216 may be indents within a material of sole 200 that may provide a channel between two or more components of sole 200, such as, but not limited to, a processor and a sensor. In some embodiments, electrical connection pathways 216 may connect a processor of sole 200 (such as the processor 108 of FIG. 1) to a wireless communication unit (such as the wireless communication unit 120 of FIG. 1).

Referring now to FIG. 2B, a top down view of a portion of sole 200 with sensor array 220 is shown. Sensor array 220 may be the same as sensor array 112 described above with reference to FIG. 1. In some embodiments, sensor array 220 may include two or more sensors. Sensor array 220 may include three sensor positioned at a front end of sole 200 and two sensors (not shown) positioned at a rear end of sole 200. Sensor array 220 may be in electrical contact with one or more components of sole 200, such as a processor, via the electrical connection pathways 216. In some embodiments, sensor array 220 may be positioned below a top surface of sole 200, such that a patient's foot does not come into direct physical contact with one or more sensors of sensor array 220. For instance, sensor array 220 may be positioned on a bottom external surface of sole 200 or embedded in the sole so that sensor array 220 can measure the pressure exerted on sole 200 without the patient's foot being in direct physical contact with the sensors in the sensor array 220.

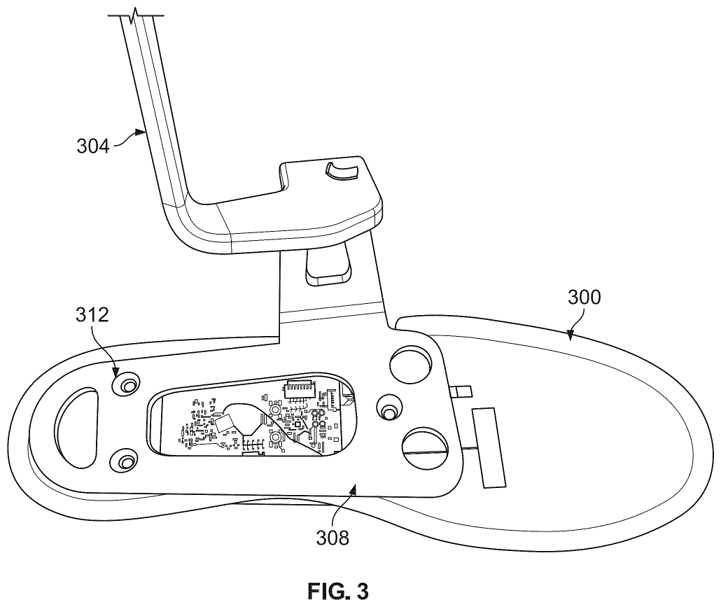

Referring now to FIG. 3, a top perspective view of a sole 300 connected to a twisted stem portion 304 is presented. According to some embodiments, sole 300 may correspond to or represent soles 200 and 104 discussed above in reference to FIGS. 1 and 2A-B. In some embodiments, sole 300 may be coupled to a connection plate 308. Connection plate 308 may be shaped as a rectangle, square, or other suitable shape. In some embodiments, connection plate 308 may be made of a rigid and/or lightweight material, such as a metal, a metal alloy, carbon fiber, etc. Connection plate 308 may include one or more connectors 312. By way of example and not limitation, connectors 312 may include screws or any suitable type of fasteners (e.g., rivets). In some embodiments, connection plate 308 may have two or more connectors 312. By way of example and not limitation, connection plate 308 can have three connectors 312, one at a front of sole 300 and two at a rear of sole 300 as shown in FIG. 3. In some embodiments, connection plate 308 may be formed from twisted stem portion 304. For instance, connection plate 308 may be formed as a unitary part with twisted stem portion 304. In some embodiments, an end of twisted stem portion 304 may be mechanically attached to a bottom of sole 300 via appropriate connections or linkages. In further embodiments, an end of twisted stem portion 304 may be mechanically attached to a side of connection plate 308 via appropriate connections or linkages. Connection of connection plate 308 with sole 300 and twisted stem portion 304 may form a continuous full length support for a patient's foot. In some embodiments, twisted stem portion 304 may house one or more electrical components of sole 300, such as, but not limited to, electrical connection wires. Twisted stem portion 304 may electrically connect sole 300 to a shin unit (not shown), according to some embodiments. For instance and without limitation, one or more electrical wires or other electrical connection devices may be housed within an interior of twisted stem portion 304 and may connect a processor of sole 300 with a processor of a shin unit described below. In some embodiments, one or more fluidic connectors, such as an air hose, may be housed within twisted stem portion 304. In some embodiments, twisted stem portion 304 may mechanically couple the sole 300 with a shin unit. Twisted stem portion 304 may be covered by a soft or padded material, such as a fabric, in some embodiments.

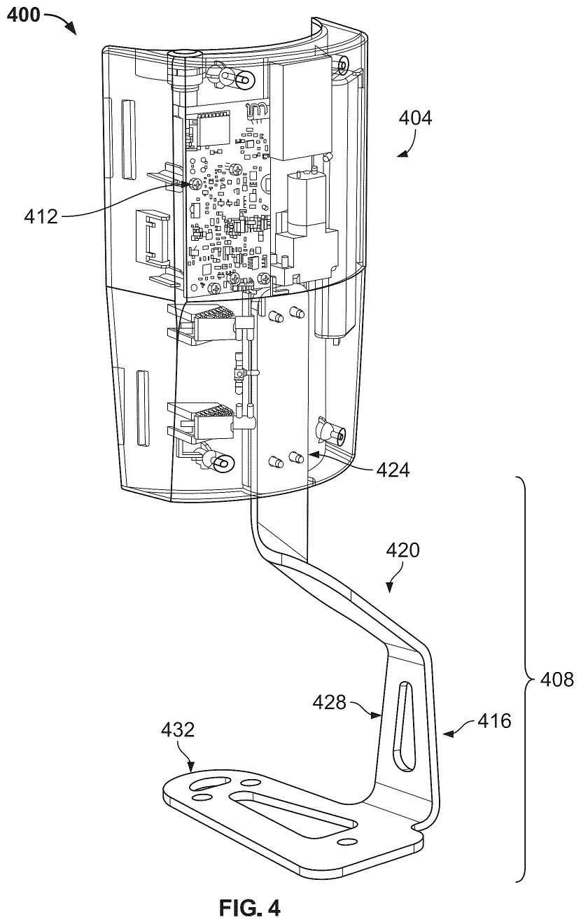

Referring now to FIG. 4, an offloader device 400 is presented. Offloader device 400 may include a shin unit 404 and a twisted stem portion 408. Shin unit 404 may include one or more circuitry components 412. Circuitry components 412 may include, but are not limited to, processors, memories, sensors, power sources, wireless communication units, pneumatic devices, air pumps, pressure relief valves, and/or other components. A processor of shin unit 404 may be configured to communicate with one or more computing devices via a wireless connection. For instance and without limitation, a processor of shin unit 404 may be configured to communicate with the processor 108 of sole 104 shown FIG. 1. Sole 104 may communicate sensor data, patient data 116, and/or other data to the processor of shin unit 404. In some embodiments, shin unit 404 may be configured to provide power for the components of sole 104. In alternative embodiments, sole 104 and shin unit 404 may each have their own respective power sources.

Sensors of shin unit 404 may include similar or additional sensors to sole 104. For instance, sensor of shin unit 404 may include accelerometer, gyroscopes, force sensors, and/or other types of sensors. Shin unit 404 may be configured to detect and communicate sensor data with one or more external computing devices, such as a smartphone of a patient, a computing device of a medical professional, and/or other devices. In some embodiments, shin unit 404 may communicate sensor data to processor 108 of sole 104 as described above with reference to FIG. 1. In other embodiments, processor 108 of sole 104, may communicate sensor data to a processor of shin unit 404. Shin unit 404 may include one or more speakers that are activated by the processor of shin unit 404 and are configured to communicate an audible output to the patient. Audible outputs may include, but are not limited to, alerts, positive feedback, medical instructions, warning messages, and/or other forms of verbal communication or audible sounds. By way of example and not limitation, audible outputs may be triggered by the processor of shin unit 404 and/or the processor 108 of sole 104. Examples of verbal prompts that can be used as audible outputs include one or more words and/or sentences, such as “sole misaligned”, “shin unit not worn properly”, “battery power low”, “please rest your foot”, “you have completed the resting phase for today, you may now walk up to 1,000 steps”, and the like. In some embodiments, audible outputs from the one or more speakers of shin unit 404 may include noises, music, tones, and/or other non-verbal communication. For instance and without limitation, audible outputs of shin unit 404 may include beeping, musical tones, chirps. Additionally, audible outputs of shin unit 404 may be in any language, without limitation. In some embodiments, libraries of verbal prompts in various languages used to generate audible outputs can be stored in the memory of shin unit 404 and/or of sole 104.

Audible outputs of shin unit 404 may include one or more commands. For instance and without limitation, audible outputs of shin unit 404 may include commands directed to the patient with respect to one or more parts of the patient's foot. For example, the audible outputs of shin unit 404 may direct the patient to align the sole and/or shin unit 404, change the angular positioning of his/her foot, reduce the temperature of the sole, and/or other commands. Audible outputs of shin unit 404 may include positive feedback, such as words and/or sentences of encouragement relating to a patient's compliancy with a therapy regime. As a non-limiting example, positive feedback may include “you're doing great! Keep resting your foot”, “way to go!” and/or other forms of positive feedback. Audible outputs of shin unit 404 may include custom messages received by a medical professional. For instance and without limitation, a medical professional may instruct the processor of shin unit 404 and/or sole 104 (e.g., via an external computing device) to generate an audio signal or audio verbal prompts for the patient to hear. In some embodiments, a processor in shin unit 404 and/or in sole 104 may be operable to send text messages to an external device operated by a medical professional, a patient, or other individual through Wi-Fi, Bluetooth, cellular communications, other communication protocols, or any combination thereof. For instance, a processor of shin unit 404 and/or of sole 104 may be operable to send text messages related to the patient's compliance with a therapy regime, sensor data, instructions from a medical professional, and/or other data to the patient's mobile device.

Referring still to FIG. 4, twisted stem portion 408 may include a vertical base section 416, a middle angled section 420, and a vertical upper end section 424. Base section 416 may angled vertically at about a 90 degree angle relative to the ground. Middle section 420 may be angled towards shin unit 404 at an angle of about 25 degrees with respect to vertical upper end section 424. Vertical upper end section 424 may be vertically aligned, like base section 416, and may be angled at about 90 degrees relative to the ground. Base section 416 may include hole 428. Hole 428 may allow for breathability of a shoe or portion thereof. Middle section 420 may be twisted away from vertical upper end section 424. In some embodiments, middle section 420 may laterally cross over a portion of a patient's leg. For instance, in some embodiments, twisted stem portion 408 may be twisted towards a front of a tibia of a patient. Twisted stem portion 408 may be designed to tri-axially immobilize a patient's foot. For instance, twisted stem portion 408 may prevent movement of an ankle of a patient. A patient's foot may be tri-axially immobilized, in an embodiment, by locking twisted stem portion 408 into to a sole or shoe, securing the sole or the shoe to the patient's foot, and placing shin unit 404 on the patient's leg.

In some embodiments, twisted stem portion 408 may link the shin unit 404 to the connection plate 432. Connection plate 432 may be the same as connection plate 308 as described above with reference to FIG. 3, without limitation. Vertical upper end section 424 of twisted stem portion 408 may be connected to a portion of shin unit 404 through one or more screws or fasteners, in some embodiments. In some embodiments, shin unit 404 may feature a slot or a cavity designed to receive the vertical upper end section 424 of twisted stem portion 408 so that the vertical upper end section end 424 may slide into the slot or cavity of shin unit 404.

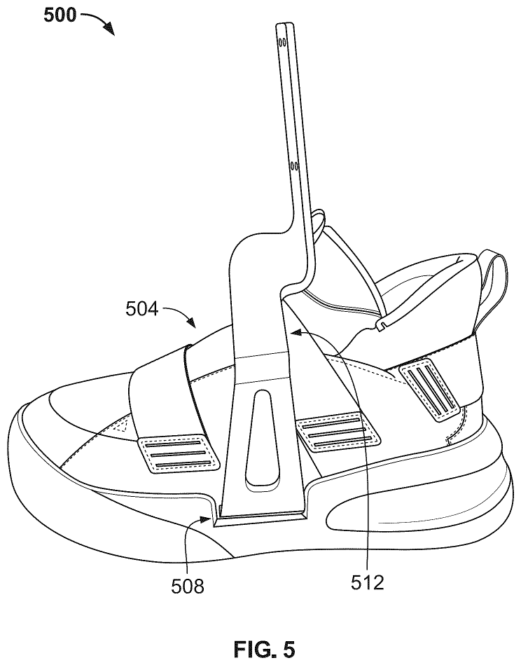

Referring now to FIG. 5, a side view of a system 500 for a footwear implemented in a shoe 504 is presented. Shoe 504 may include connection port 508. According to some embodiments, connection port 508 may be a receiving opening (e.g., a hole or a slot) on a side surface of shoe 500 that may allow twisted stem portion 512 to be removably inserted into shoe 500. According to some embodiments, twisted stem portion 512 may correspond to or represent the twisted stem portion 408 shown FIG. 4. Connection port 508 may allow a patient to introduce or add a shin unit, such as the one described above with reference to FIG. 4, in combination with a sole, such as the one described above with reference to FIGS. 1 and 2A-B. In some embodiments, a medical provider may remove the twisted stem portion 512 from the connection port 508 of shoe 500, which may allow a patient to use the sole, such as shole 104, 200, and 300 discussed above, independently from a shin unit.

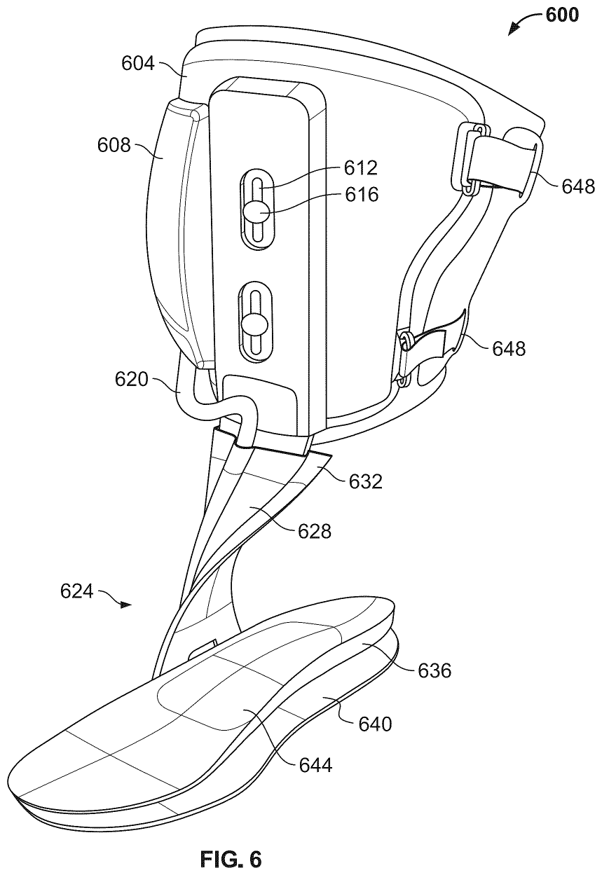

Referring now to FIG. 6, an illustration of an embodiment of a system for footwear 600 is presented. System 600 may include a shin support structure 604. Shin support structure 604 may be connected to a sock 636 (otherwise known as an innersole) via an offloading element 624 (or offloader). The sock 636 may be disposed on top of a plate portion 640 of the offloading element 624. A lower half of system 600 may be configured to be disposed within a boot (not shown). A lower half of the offloader element 624 may be affixed to a boot, as described in detail below.

Shin support structure 604 may include one or more devices for mechanically fixing the offloading element 624 to the shin support structure 604, such as, but not limited to, screws, straps, and/or other devices. For instance one or more screws 616 may be disposed within a slide fixing 612 such that the location of the one or more screws 616 can be adjusted within a range of different positions, the screws 616 engaging with the offloading element 624 so as to affix the offloading element 624 to the shin support structure 604. In some embodiments, module 608 and/or other portions of system 600 may be connected to shin support structure 604 via straps 648. In some embodiments, system 600 may include two straps 648. In other embodiments, system 600 may include three or more straps 648. Straps 648 may be Velcro, magnetic, or other variations of straps.

Shin support structure 624 may include a module 608. Module 608 may include one or more components of a blood flow stimulation mechanism. A blood flow stimulation mechanism may include one or more devices configured to stimulate blood flow in a user's foot. Module 608 may be connected to conduit 620. Conduit 620 may be connected to a bladder 644. Bladder 644 may be disposed within or on the sock 636. Bladder 644 may be inflated with a fluid (e.g., a gas or a liquid) to provide pressure or support to one or more parts of a patient's foot. For instance, bladder 644 may abut a plantar plexus or a medial plantar arch of a foot of a patient. In some embodiments, shin support structure 624 and module 608 may be as described above with reference to FIG. 4, without limitation.

Conduit 620 may be made of a non-expandable material that may allow for a reduction in energy loss in blood flow stimulation mechanisms described throughout this disclosure. Conduit 620 may travel from module 608 to bladder 644. In some embodiments, conduit 620 may travel alongside offloading element 624, such as alongside twisted stem portion 628.

In some embodiments, module 608 may include additional components such as a pump (e.g., an electric pump) powered by a battery. A pump of module 608, which is not shown in FIG. 6, can be fluidically connected via conduit 620 to bladder 644 such that bladder 644 can be inflated or deflated when the pump is operated. By way of example and not limitation, the fluid used to inflate or deflate the bladder can be a non-toxic gas or liquid, such as air, argon, helium, oil, water, and the like. In some embodiments, module 608 may include a fluid reservoir, which may or may not be expandable. A fluid reservoir may be configured to store a volume of the fluid, such as pressurized air. Module 608 may be equipped with one or more valves in fluidic communication with conduit 620 and/or the electric pump. One or more valves may be configured to regulate the flow of the fluid from a reservoir of module 608 to bladder 644. In some embodiments, module 608 may include an air inlet positioned on a side of shin support structure 604 (not shown) and configured to intake atmospheric air into a fluid reservoir of shin support structure 604.

Referring still to FIG. 6, the pump of module 608 may be in fluidic communication with an air inlet of shin support structure 604 and in fluidic communication with a fluid reservoir of shin support structure 604 via a first valve of shin support structure 604. The fluid reservoir of shin support structure 604 may be in fluidic communication with bladder 644 via a second valve of module 608 and conduit 620. A controller of shin support structure 604, which may be located in module 608, may control the pump, the first valve, and/or the second valve of shin support structure 604. In some implementations, the first valve of shin support structure 604 may be a one-way valve and may be mechanically or electrically activated. The controller of shin support structure 604 may provide air to a fluid reservoir of shin support structure 604 via the pump. The fluid reservoir of shin support structure 604 may become pressurized in some embodiments. A second valve of shin support structure 604 may enable short bursts of pressurized air to flow from a fluid reservoir of shin support structure 604 into bladder 644 via conduit 620. The controller of shin support structure 604 may control a second valve of shin support structure 604, in some embodiments. Shin support structure 604 may include a third valve, which may provide controlled deflation of bladder 644. The third valve of shin support structure 604 may be a pressure relief valve that may assist in preventing over pressurization of bladder 644. The fluid reservoir of shin support structure 604 may be pressurized to produce a sharp rise in pressure at bladder 644 once a second valve is opened, which may cause bladder 644 to undergo rapid inflation. The third valve of shin support structure 604 may be configured like a bleed valve to provide rapid deflation of bladder 644. In some embodiments, rapid deflation of bladder 644 may occur within 3 to 4 seconds. In some embodiments, shin support structure 624 and module 608 may be as described above with reference to FIG. 4, without limitation.

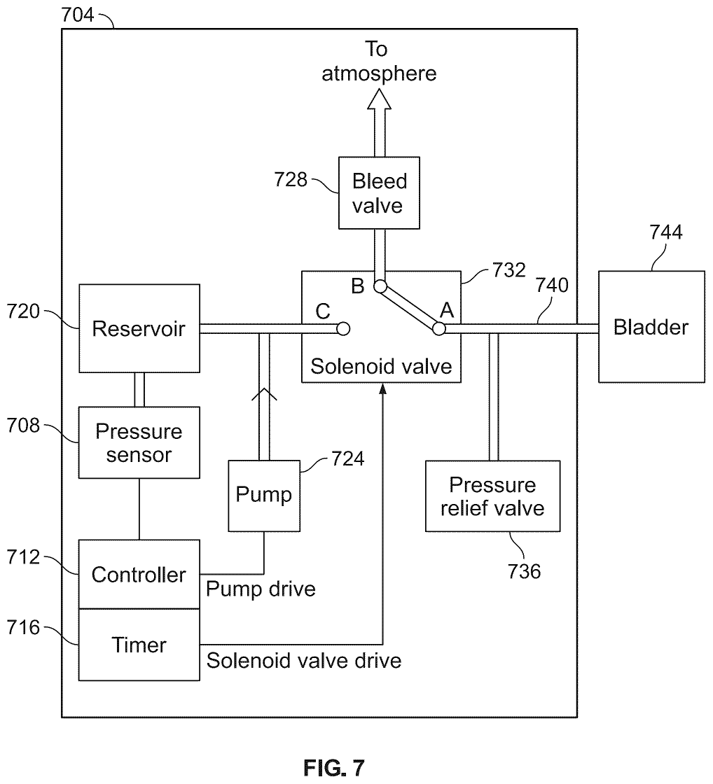

Rapid inflation of bladder 644 followed by rapid deflation of bladder 644 may maximize a blood flow promoting the effect of bladder 644. A rapid action of bladder 644 may deliver a spike rather than a hump of blood flowing back up the veins of a patient, which may allow the blood to travel further upwards towards the heart of the patient. In some embodiments, the second valve of shin support structure 604 may be open for a long period so as to enable a portion of bladder 644 abutting the plantar plexus/medial plantar arch to compress the plantar plexus veins located in the plantar arch region of a foot of a patient, such that the subdermal veins at least partially close, thus forcing the blood contained therein to return towards the abdomen. A fluidic circuit that can support the operations of components in module 608, as described above, is discussed further below in reference to FIG. 7.

In referring to FIG. 6, offloading element 624 may include twisted stem portion 628 and a plate portion 640. The twisted stem portion 628 of the offloading element 624 may be attached to the shin support structure 604. The twisted stem portion 628 of the offloading element 624 may twist from a position in front of the tibia of a wearer of the device, at its end closest to the shin support structure 604, to a position flush with the side of the sole of foot of the wearer of the device, as depicted in FIG. 6. A padded fabric sleeve 632 may be disposed around an exterior surface of the twisted stem portion 628 of the offloading element 624. For instance, in some embodiments, sleeve 632 may encapsulate an entirety of twisted stem portion 628. The plate portion 640 of the offloading element 624 may include one or more holes which may allow for plate portion 640 to be securely affixed into a bottom inner surface of a boot. Sock 636 may be placed on top of the plate portion 640 of the offloading element 624. In some embodiments, the lower half of the twisted stem portion 628, the entirety of the plate portion 640 of the offloading element 624, and the sock 636, may be disposed within a boot enclosure (not shown).

A wearer of system 600 may wear a boot, to which the plate portion 640 of the offloading element 624 may be securely fastened, on their foot, and may affix the shin support structure 604 below their knee, so that pressure can be transferred from their foot to their shin when standing on the leg on which system 600 is worn. Advantageously, a patient wearing system 600 can still flex his/her knee.

With continued reference to FIG. 6, one or more sensors, such as sensor array 112 as described above with reference to FIG. 1, may be disposed underneath sock 636. In some embodiments, one or more sensors may be disposed between sock 636 and plate portion 640. As discussed above, the one or more sensors are not in direct physical contact with a wearer's foot so that the wearer cannot feel the underlying sensors. In some embodiments, one or more sensors may be attached to plate portion 640, as shown above with reference to FIGS. 2A-B. An innersole, such as sock 636, may sit on top of one or more sensors attached to plate portion 640. Since shin support structure 604 may be attached to plate portion 640 in some embodiments via twisted stem portion 628, one or more sensors may be disposed around a connection of twisted stem portion 628 and plate portion 640 to accommodate the shape of the twisted stem portion 628 connecting to plate portion 640.

Referring now to FIG. 7, a block diagram of an exemplary fluidic circuit 700 that may be implemented with system 600 is shown. The depiction of fluidic circuit 700 in FIG. 6 is not limiting, and variations of fluidic circuit 700 are within the spirit and the scope of this disclosure. According to some embodiments, the fluidic circuit 700 can include module 704, which further includes a pump 724, a controller 712, a timer 716 in communication with controller 712, a reservoir 720, a pressure sensor 708, a solenoid valve 732, a pressure relief valve 736, and a bladder 744. In some embodiments, the pressure relief valve 736 can keep a pressure inside the bladder 744 below a predetermined threshold, such as, but not limited to, about 3.4 psi. Module 704 may include conduit 740 which may connect solenoid valve 732 to bladder 744. Bladder 744 may be located in a sole of a shoe, as discussed above in reference to bladder 644. Controller 712 can be communicatively coupled to pump 724, solenoid valve 732, and/or pressure sensor 708.

As shown in FIG. 7, pump 724 can be in fluidic communication with reservoir 720 and/or solenoid valve 732. Pressure sensor 708 can be communicatively coupled to reservoir 720 to provide pressure readings from reservoir 720. For instance, pressure sensor 708 may be configured to detect when a pressure of reservoir 720 reaches a predetermine value or is below a predetermined value, such as, but not limited to, about 5 psi. Based on the readings from pressure sensor 708, controller 712 may operate pump 724 by turning it ON and/or OFF.

According to some embodiments, solenoid valve 732 may be configured to switch between two configurations. For instance, solenoid valve 732 may be configured to switch between an A-B configuration, in which the bladder 744 is in fluidic communication with bleed valve 628, and an A-C configuration, in which bladder 744 is in fluidic communication with reservoir 720.

Referring now to FIG. 8, a screenshot of an exemplary graphical user interface (GUI) 800 is presented. GUI 800 may include a graph 804. By way of example and not limitation, graph 804 may be a bar graph, a line graph, or heat map. In some embodiments, graph 804 may have an x-axis representing time, such as intervals of time throughout a day (e.g., 10 minute intervals, 20 minute intervals, 30 minute intervals, hourly intervals, and the like). In some embodiments, graph 804 may have a y-axis, which may represent pressure values or other sensor output data from the sensor array 112 shown in FIG. 1. In some implementations, graph 804 may show pressure data across multiple zones of a patients foot, such as, but not limited to, a first zone 808, a second zone 812, a third zone 816, a fourth zone 820, and/or a fifth zone 824. In some embodiments, graph 804 may include data from five or more zones of a patient's foot.

GUI 800 may include a filtering table 828. Filtering table 828 allows one or more parameters to be included or removed from graph 804. For instance, and without limitation, parameters of filtering table 828 may include sensor metrics, such as pressure, temperature, humidity, etc. In some embodiments, parameters of filtering table 828 may include one or more channels representing a zone of a patient's foot. A user, such as a medical professional, may interact with GUI 800 to plot and interpret sensor data generated by any sensor described herein.

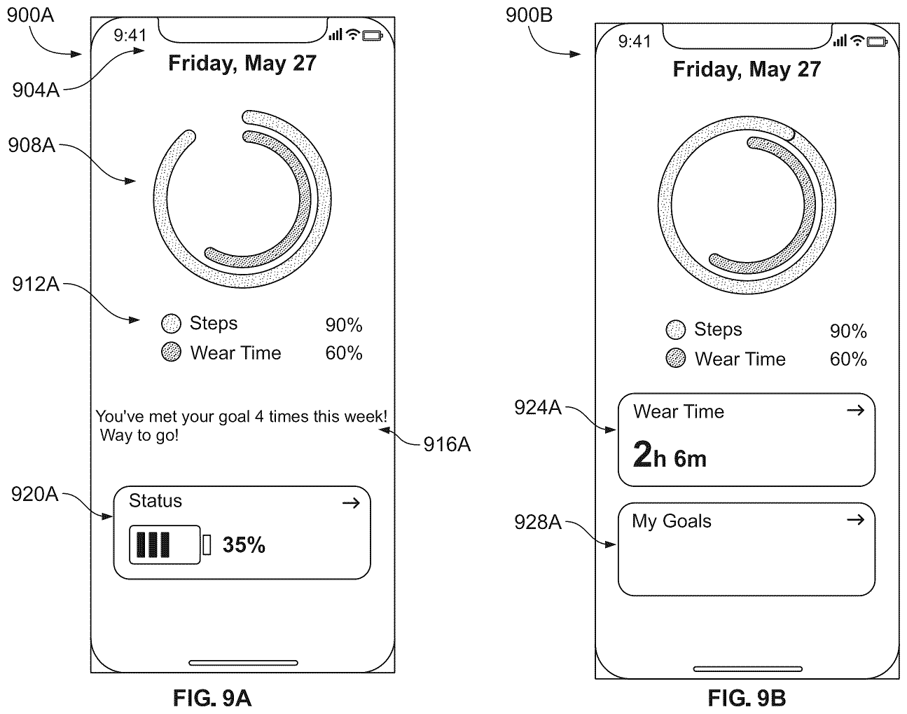

Referring now to FIG. 9A, an exemplary graphical user interface (GUI) 900A is presented. GUI 900A may be displayed on a smartphone, monitor, tablet, or other display device. In some embodiments, GUI 900A may be generated based on data gathered by processor 108 shown in FIG. 1. For instance in some embodiments, a display device may be in communication with processor 108 through wireless communication unit 120. Data may be communicated by processor 108 via wireless communication unit 120 to one or more display devices. Data may include data generated by sensor array 112 and/or calculations performed by processor 108. For instance, data communicated to a display device that may present GUI 900A may include, but is not limited to, pressure data, temperature data, pedometer data, compliancy data, power data, and/or other data as described throughout this disclosure, without limitation.

Referring still to FIG. 9A, GUI 900A may include display device statuses 904A. Display device statuses 904A may include, but are not limited to, dates, times, cellular signal strength, Wi-Fi signal strength, power level, and/or other statuses of a display device, such as a smartphone. GUI 900A may include goal ring 908A. Goal ring 908A may be a circular ring that may represent one or more parameters of a therapy regime for a patient. A circumference of goal ring 908A may indicate a percent completion of one or more parameters of a therapy regime for a patient. For instance, a goal ring 908A with a partially filled circumference may represent a partial completion of one or more parameters of a therapy regime expressed as a percentage of completion of the one or more parameters of a therapy regime. By way of example and not limitation, a patient may complete about 90% of a quantity of steps in a therapy regime to which goal ring 908A may have a circumference that may be about 90% complete with relation to a full circle or ring. In some embodiments, goal ring 908A may include a second goal ring that may be concentric with the first goal ring 908A. The second goal ring may represent one or more parameters of a therapy regime that are different from the ones represented by the first goal ring 908A. As a non-limiting example, the second goal ring may represent a total wear time of a device in relation to a wear time set by a therapy regime. A completion of a circumference of the second goal ring may represent a total wear time of a device of a patient. As a non-limiting example, a patient may wear the device for 60% of a total time prescribed by a therapy regime, which may be represented by about a 60% completion of a circumference of the second goal ring. In some embodiments, goal rings in FIG. 9A may include any appropriate number of concentric rings, each concentric ring representing a respective parameter of a therapy regime.

Still referring to FIG. 9A, GUI 900A may include legend 912A. Legend 912A may include icons whose colors correspond to respective colors of the one or more rings of goal ring 908A. Icons of legend 912A may be circular, square, rectangular, or have any suitable shape. A first goal ring of goal ring 908A may be color coded to a first icon of legend 912A. As a non-limiting example, a first icon of legend 912A may be purple and a first ring of goal ring 908A may be purple. Continuing this non-limiting example, a second icon of legend 912A may be green and a second ring of goal ring 908A may be green. One or more icons of legend 912A may be displayed with text. For instance, text may indicate what each icon represents. As a non-limiting example, a first icon of legend 912A may be displayed next to text of “Steps” and a second icon of legend 912A may be displayed next to text of “Wear Time.” Legend 912A may include one or more numerical values displayed adjacent to one or more icons. For instance, numerical values may include quantities of steps, total wear time, percent completion of one or more parameters of a therapy regime, or other numerical values. In some embodiments, legend 912A may be displayed underneath goal ring 908A. Goal ring 908A and legend 912A may be displayed on a center of GUI 900A or in any suitable location without limitation.

In some embodiments, GUI 900A may include compliancy message 916A. Compliancy message 916A may be a text box that may include one or more characters, strings, words, and/or other textual data. Compliancy message 916A may display one or more messages relating to a completion of one or more parameters of a therapy regime for a patient. For instance, compliancy message 916A may include information relating to an amount of steps taken, a total wear time of a device, a rest time of a leg and/or foot of a patient, and/or other parameters. In some embodiments, compliancy message 916A may indicate a completion of one or more parameters of a therapy regime of a patient. For instance, compliancy message 916A may indicate a completion of one or more parameters per day, per week, per month, or a completion of one or more parameters over other time periods, without limitation. In some embodiments, compliancy message 916A may display text indicative of remaining completion of parameters of a therapy regime, such as a remaining amount of steps to take, a remaining amount of wear time of a device, a remaining amount of rest time, and/or other parameters of a therapy regime. Compliancy message 916A may be generated based on data communicated by processor 108 via wireless communication module 120. In some embodiments, data may be communicated from processor 108 via wireless communication module 120 to an external computing device, which may generate one or more portions of GUI 900A.

Still referring to FIG. 9A, GUI 900A may include device status icon 920A. Device status icon 920A may include a pictorial icon, such as a battery, foot, thermometer, or other icon. Device status icon 920A may display various parameters relating to a status of a device, such as the device described above with reference to FIGS. 1-5. For instance, device status icon 920A may include a battery icon showing a percent charge of a battery of a device. Device status icon 920A may include a thermometer icon showing a temperature of a device. Device status icon 920A may include a pump icon showing a pressure of a device. In some embodiments, device status icon 920A may include a numerical value that may be displayed next to device status icon 920A. For instance, a numerical value may be a percentage value, a temperature value, a pressure value, or a combination thereof. In some embodiments, device status icon 920A may be displayed within a display box, which may be contrasted to a background of GUI 900A. For instance, a display box of device status icon 920A may be shaded lightly gray or other colors. In some embodiments, device status icon 920A may be interactive. For instance a patient may click on, tap, or otherwise interact with device status icon 920A through a display device that may be displaying GUI 900A. Interaction with device status icon 920A may cause GUI 900A to animate to a new page which may provide more detail on a status of a device to a patient. For instance, a patient may tap on device status icon 920A which may cause a pop-up window to be displayed on GUI 900A. A pop-up window displayed through interaction of device status icon 920A may include text and/or numerical values that may display device stats data such as total run time, battery percentage, pressure values, time since last worn, remaining predicted battery life, a level of fit a patients foot and/or leg may have to the device, and/or other data.

Referring now to FIG. 9B, GUI 900B is displayed. GUI 900B may be similar to GUI 900A described above with reference to FIG. 9A. In some embodiments, GUI 900B may include parameter tracker 924A. Parameter tracker 924A may be a display box that may be contrasted to a background of GUI 900B. Parameter tracker 924A may display one or more numerical values relating to a completion of one or more parameters of a therapy regime. For instance and without limitation, parameter tracker 924A may display numerical values and/or text representing a total wear time, a total number of steps, a total rest time, a total pump/bladder usage, and/or other parameters. A patient may click, tap, or otherwise interact with parameter track 924A which may cause GUI 900B to animate to a different screen or display a pop-up window. Another screen or pop-up window displayed b GUI 900B in response to a patient interacting with parameter track 924A may include additional detail of one or more parameters of a therapy regime, such as a total wear time of a device over a period of time, an average wear time per day, an initial start of a wear time, a projected end time of a wear time, and/or other details of one or more other parameters. In some embodiments, GUI 900B may include a goals button 928A. Goals button 928A may be a rectangular box that may be about a same size as a box of parameter tracker 924A. Goals button 928A may include text that displays one or more goals of a patient. In some embodiments, goals button 928A may display text reading “My Goals.” A patient may interact with goals button 928A which may cause GUI 900B to animate to another screen or display a pop-up window that may detail one or more goals of a patient with respect to one or more parameters of a therapy regime. For instance, a list of one or more parameters of a therapy regime may be displayed alongside one or more numerical values representing completion of the one or more parameters, such as, but not limited to, total wear time of a device, total steps taken, total rest time, total pressure, and/or other parameters.

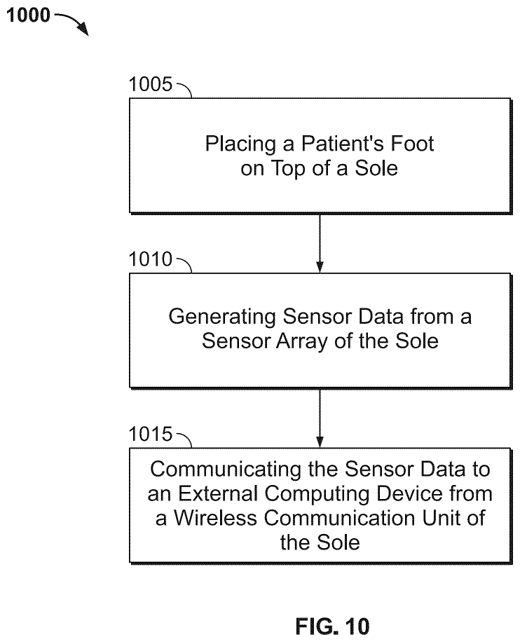

Referring now to FIG. 10, a remote-patient monitoring method 1000 is presented. According to some embodiments, method 1000 may be implemented with the system and components discussed above in connection to FIGS. 1, 2A-B, 3, 4, 5, and 6. Method 1000 begins with step 1005, in which a prospective patient places his/her foot on the sole, such as sole 104, 200, 300, or 636 (e.g., by wearing shoe 504 shown in FIG. 5). The sole may include one or more sensors, such as the sensor array 112 described above. The patient may place his/her foot on a top surface of the sole, as described above with reference to FIG. 3.

Method 1000 continues with step 1010 where sensor data are being generated from a sensor array in the sole, much like sensor array 112 in sole 104. As discussed above, the sensor array may include one or more sensors (e.g., pressure sensors, gyroscopes, humidity sensors, temperature sensors, pedometers, and/or other sensors, etc.), and the sensor data may include pressure data, temperature data, a number of steps taken, humidity data, periods of use, periods of rest, and/or other types of sensor data. In some embodiments, a processor of the sole, like processor 108 of sole 104, may be configured to generate a pressure multi-zone mapping of the patient's foot. A pressure multi-zone mapping of the patient's foot may include five or more separate zones.

Method 1015 concludes with step 1015 where the generated sensor data are transmitted to an external computing device, such as external computing device 124. As discussed above, sensor data may be broadcasted via a wireless communication unit, such as wireless communication unit 120, using any type of suitable communication protocol, such as Wi-Fi communication protocols, Bluetooth communication protocols, cellular communication protocols, or other types of wireless communication protocols. In some embodiments, the processor of the sole, such as processor 108 of sole 104, may communicate with one or more external computing devices, such as external computing device 124, through a wireless communication unit, such as wireless communication unit 120. External computing devices may include, but are not limited to, smartphones, tablets, laptops, desktops, servers, and/or other devices. In some embodiments, an external computing device may be used by a medical professional who may interpret the sensor data.

In some embodiments, method 1000 further includes comparing the sensor data to one or more parameters of a therapy regime to determine a compliancy of a patient. In some embodiments, method 1000 further includes producing by a shin unit an audible output for the patient to hear based on the transmitted sensor data. In some embodiments, method 1000 further includes adjusting one or more parameters of a therapy regime based on the transmitted sensor data. In some embodiments, method 1000 further includes communicating one or more text messages from a medical professional to the shin unit for the patient to hear via one or more speakers of the shin unit when the shin unit is communicatively coupled, via a network connection, to an external computing device operated by the medical professional.

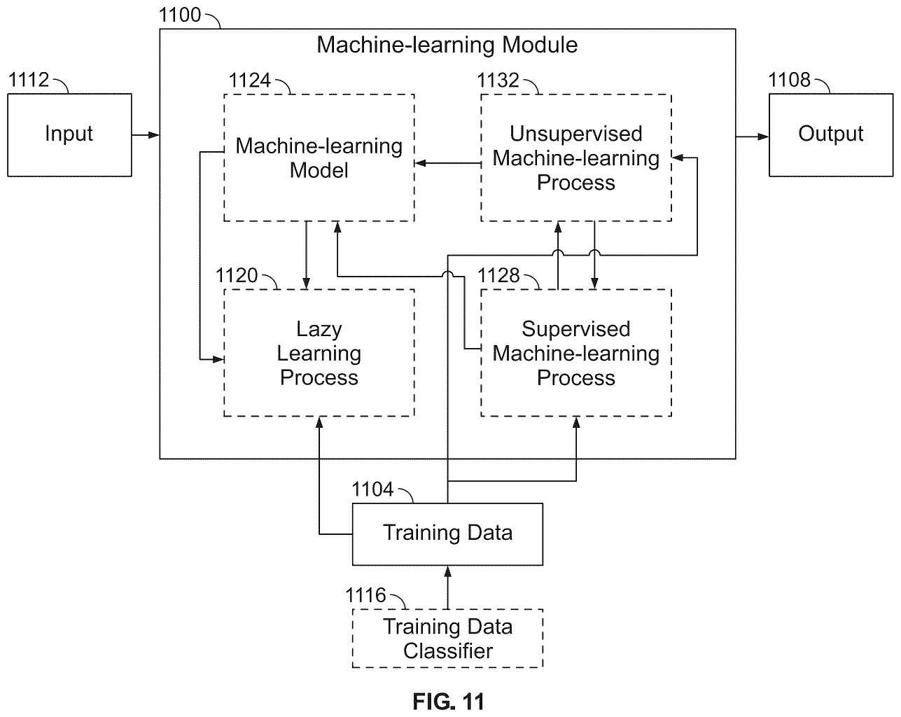

Referring to FIG. 11, an exemplary machine-learning module 1100 may perform machine-learning process(es) and may be configured to perform various determinations, calculations, processes and the like as described herein using one or more machine-learning processes.

Machine learning module 1100 may utilize training data 1104. For instance, and without limitation, training data 1104 may include a plurality of data entries, each entry representing a set of data elements that were recorded, received, and/or generated together. Training data 1104 may include data elements that may be correlated by shared existence in a given data entry, by proximity in a given data entry, or the like. Multiple data entries in training data 1104 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1104 according to various correlations. Correlations may indicate causative and/or predictive links between categories of data elements, which may be modeled as relationships such as mathematical relationships by machine-learning processes as described in further detail below. Training data 1104 may be formatted and/or organized by categories of data elements. Training data 1104 may, for instance, be organized by associating data elements with one or more descriptors corresponding to categories of data elements. As a non-limiting example, training data 1104 may include data entered in standardized forms by one or more individuals, such that entry of a given data element in a given field in a form may be mapped to one or more descriptors of categories. Elements in training data 1104 may be linked to descriptors of categories by tags, tokens, or other data elements. Training data 1104 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 11, training data 1104 may include one or more elements that are not categorized. Uncategorized data of training data 1104 may include data that may not be formatted or containing descriptors for some elements of data. In some embodiments, machine-learning algorithms and/or other processes may sort training data 1104 according to one or more categorizations. Machine-learning algorithms may sort training data 1104 using, for instance, natural language processing algorithms, tokenization, detection of correlated values in raw data and the like. In some embodiments, categories of training data 1104 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries in an automated fashion may enable the same training data 1104 to be made applicable for two or more distinct machine-learning algorithms as described in further detail below. Training data 1104 used by machine-learning module 1100 may correlate any input data as described in this disclosure to any output data as described in this disclosure, without limitation.

Further referring to FIG. 11, training data 1104 may be filtered, sorted, and/or selected using one or more supervised and/or unsupervised machine-learning processes and/or models as described in further detail below. In some embodiments, training data 1104 may be classified using training data classifier 1116. Training data classifier 1116 may include a classifier. A “classifier” as used in this disclosure is a machine-learning model that sorts inputs into one or more categories. Training data classifier 1116 may utilize a mathematical model, an artificial neural network, or a program generated by a machine learning algorithm. A machine learning algorithm of training data classifier 1116 may include a classification algorithm. A “classification algorithm” as used herein is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine-learning module 1100 may generate a classifier, such as training data classifier 1116 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1116 may classify elements of training data to one or more faces.

Still referring to FIG. 11, machine-learning module 1100 may be configured to perform a lazy-learning process 1120 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1104. Heuristic may include selecting some number of highest-ranking associations and/or training data 1104 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described herein, including lazy learning applications of machine-learning algorithms as described in further detail below.

Still referring to FIG. 11, machine-learning processes as described herein may be used to generate machine-learning models 1124. A “machine-learning model” as used herein is a mathematical and/or algorithmic representation of a relationship between inputs and outputs, as generated using any machine-learning process including without limitation any process as described above, and stored in memory. For instance, an input may be sent to machine-learning model 1124, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, machine-learning model 1124 may be generated by creating an artificial neural network, such as a convolutional neural network comprising an input layer of nodes, one or more intermediate layers, and an output layer of nodes. Connections between nodes may be created via the process of “training” the network, in which elements from a training data 1104 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 11, machine-learning algorithms may include supervised machine-learning process 1128. A “supervised machine learning process” as used herein is one or more algorithms that receive labelled input data and generate outputs according to the labelled input data. For instance, supervised machine learning process 1128 may include sensor data as described above as inputs, compliancy scores as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 1104. Persons skilled in the art, upon reviewing the entirety of this disclosure, will be aware of various possible variations of at least a supervised machine-learning process 1128 that may be used to determine relation between inputs and outputs. Supervised machine-learning processes may include classification algorithms as defined above.

Further referring to FIG. 11, machine learning processes may include unsupervised machine-learning processes 1132. An “unsupervised machine-learning process” as used herein is a process that calculates relationships in one or more datasets without labelled training data. Unsupervised machine-learning process 1132 may be free to discover any structure, relationship, and/or correlation provided in training data 1104. Unsupervised machine-learning process 1132 may not require a response variable. Unsupervised machine-learning process 1132 may calculate patterns, inferences, correlations, and the like between two or more variables of training data 1104. In some embodiments, unsupervised machine-learning process 1132 may determine a degree of correlation between two or more elements of training data 1104.

Still referring to FIG. 11, machine-learning module 1100 may be designed and configured to create a machine-learning model 1124 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, according to some embodiments.

Continuing to refer to FIG. 11, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes, such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Representative embodiments are described above. It will be understood that reasonable equivalents to the embodiments described above, or to the elements of the embodiments described above, are consistent with practicing the present invention and included in the present disclosure.

1. An apparatus for footwear, comprising:
a sole adapted for insertion into the footwear;
a sensor array disposed below a top surface of the sole, wherein the sensor array is configured to generate sensor data for a foot of a patient wearing the footwear;
a processor configured to initialize reference values of the sensor array to be patient-specific; receive the sensor data from the sensor array; and communicate the sensor data to an external computing device;
an inflatable bladder;
a connection plate that facilitates coupling the sole to a shin unit and provides for fluidic connection between the inflatable bladder and a conduit;
a shin unit processor in communication with the processor; and
a fluidic circuit in fluidic communication with the inflatable bladder via a conduit, wherein the shin unit processor is configured to operate the fluidic circuit to adjust a pressure of the inflatable bladder to stimulate blood flow in the foot of the patient.

2. The apparatus of claim 1, wherein the processor is further configured to generate a pressure multi-zone mapping for the foot based on the sensor data.

3. The apparatus of claim 2, wherein the pressure multi-zone mapping includes at least 5 zones of the foot.

4. The apparatus of claim 1, wherein the processor is further configured to monitor a compliance of the person wearing the shoe wear with a therapy program based on a comparison between the sensor data and reference data provided by the therapy program.

5. The apparatus of claim 1, wherein the processor is further configured to count a number of steps taken by the person wearing the shoe wear over a period of time based on the sensor data.

6. The apparatus of claim 1, wherein the processor is further configured to generate alerts based on a comparison of the sensor data to a threshold value.

7. The apparatus of claim 1, wherein the sensor data comprises at least one of temperature data and humidity data.

8. The apparatus of claim 1, wherein the sensor array comprises three sensors disposed near a front portion of the sole and two sensors disposed at a rear portion of the sole.

9. The apparatus of claim 1, wherein the processor is further configured to prepare and send text messages to the external computing device.

10. The apparatus of claim 1, further comprising a power source configured to provide power to the sensor array and the processor.

11. A system for footwear, comprising:
a sole adapted for insertion into the footwear, the sole including a processor;
a sensor array configured to generate sensor data for a foot of a patient wearing the footwear, the sensor array comprising a pressure sensor, wherein the processor is configured to initialize reference values of the sensor array to be patient-specific;
a connection plate; and
an offloading device comprising:
a twisted stem portion mechanically connected to the connection plate; and
a shin unit mechanically connected to the sole through the twisted stem portion,
wherein a processor of the shin unit is configured to receive the sensor data from the processor of the sole, and wherein the twisted stem portion facilitates constraining a tri-axis ankle movement of the foot of the person when the twisted stem portion is secured into a midsole of the shoe wear.

12. The system of claim 11, wherein the shin unit comprises:
a wireless communication unit;
one or more speakers; and
a processor in communication with the wireless communication unit and the one or more speakers.

13. The system of claim 12, wherein the processor of the shin unit is configured to:
receive the sensor data generated by the sensor array; and
control the one or more speakers to produce an audible output based on the sensor data.

14. The system of claim 13, wherein the audible output comprises a verbal command, a sound, or a combination thereof.

15. The system of claim 11, wherein the sensor array of the sole further comprises a gyroscope.

16. The system of claim 15, wherein the sole further comprises a processor configured to receive the sensor data from the sensor array and to determine a relative position of the foot based on data generated by the gyroscope.

17. The system of claim 16, wherein the processor is further configured to determine a compliancy to a therapy program followed by the person wearing the shoe wear based on the sensor data.

18. The system of claim 16, wherein the processor is further configured to determine when the person is using the sole based on the sensor data.

19. The system of claim 16, wherein the processor is further configured to generate a pressure multi-zone mapping of the foot based on the sensor data.

20. The system of claim 19, wherein the pressure multi-zone mapping includes at least 5 zones.
